(19) United States
(12) Patent Application Publication
(10) Pub. No.: US 2017/0095592 A1
(43) Pub. Date: Apr. 6, 2017

(54) Compositions For An Injectable, In Situ Forming Neuroscaffold And Methods Of Using The Same

(71) Applicant: PixarBio Corporation, Medford, MA (US)

(72) Inventors: Jason M. Criscione, Chelmsford, MA (US); Francis M. Reynolds, Salem, NH (US); Haining Dai, Rockville, MD (US); Rebecca A. Bader, Billerica, MA (US)

(21) Appl. No.: 15/281,866

(22) Filed: Sep. 30, 2016

Related U.S. Application Data
(60) Provisional application No. 62/236,309, filed on Oct. 2, 2015.

(51) Int. Cl. A61L 27/18; A61L 27/58; A61L 27/54; A61L 27/50

(52) U.S. Cl. CPC A61L 27/18; A61L 27/50; A61L 27/58; A61L 27/54; A61L 2400/06; A61L 2430/32

(57) ABSTRACT

Disclosed are injectable, biodegradable neuroscaffolds formed in situ by self-assembling biodegradable polymeric microparticles, nanoparticles, or any combination thereof, via copper-free click chemistry or Michael-type addition coupling reactions. The injectable, biodegradable neuroscaffolds provide 3-D structural support, neuroprotection, and/or subsequent regeneration in a subject with a spinal cord injury or a focal neurological disorder.

### CROSS-REFERENCE TO RELATED APPLICATIONS

This application claims priority to U.S. Provisional Application No. 62/236,309, filed Oct. 2, 2015, the entire contents of which are incorporated herein by reference.

### TECHNICAL FIELD

The present disclosure relates to injectable compositions that form biodegradable neuroscaffolds in situ. Provided herein are compositions, methods, and kits for an injectable, in situ forming scaffold capable of providing 3-dimensional (3-D) structural support, neuroprotection and/or subsequent regeneration in a subject with a spinal cord injury or a focal neurological disorder.

### BACKGROUND

Neurological disorders and disease affecting the central and peripheral nervous systems are numerous and widespread across patient populations of the world. Most of these neurological indications are derived from nociceptive pain, neuropathic pain, neurotrauma, neuro-inflammation, neurodegenerative disease, seizure disorders, neurological autoimmunity, or neuro-oncological disease. Typically, medical intervention, consisting predominantly of neuro-stimulation, surgical tissue resection, or administration of blood-brain barrier (BBB) crossing therapeutics, is only capable of providing treatment or management options for patients. That said, the medical community continues to search for options that are capable of providing paths forward in the direction structural and functional recovery for these neurological conditions. Non-injectable, implantable neuroscaffolds have been developed and are currently in pilot clinical trials, however, these neuroscaffolds have limitations, including, necessitating invasive surgical implantation and having an inability to precisely conform to the neuroanatomical landscape of interest. Additionally, non-injectable, implantable neuroscaffolds have limitations in their ability to transplant viable cells to promote tissue regeneration that are derived from the necessity to seed the neuroscaffold with cell ex vivo prior to implantation.

### SUMMARY

Provided herein are injectable, biodegradable neuroscaffolds formed in situ by self-assembling surface-functionalized polymeric microparticles, nanoparticles, or any combination thereof, via copper-free click chemistry or Michael-type addition coupling reactions. These injectable, biodegradable neuroscaffolds are designed specifically to provide 3-D structural support and subsequent neuroprotection in a subject with a spinal cord injury or a focal neurological disorder.

Also provided herein are injectable, biodegradable neuroscaffolds formed in situ by self-assembling surface-functionalized polymeric microparticles, nanoparticles, or any combination thereof, via copper-free click chemistry or Michael-type addition coupling reactions further comprising one or more agents designed specifically to provide 3-D structural support and to enhance neuroprotection in a subject with a spinal cord injury or a focal neurological disorder.

Also provide herein are injectable, biodegradable neuroscaffolds formed in situ by self-assembling surface-functionalized polymeric microparticles, nanoparticles, or any combination thereof, via copper-free click chemistry or Michael-type addition coupling reactions further comprising cells designed specifically to provide 3-D structural support, enhance neuroprotection and promote regeneration in a subject with a spinal cord injury or a focal neurological disorder.

Methods of providing 3-D structural support, subsequent neuroprotection and/or regeneration in a subject having a spinal cord injury or a focal neurological disorder comprising administering the disclosed compositions and kits for producing the disclosed compositions are also provided.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific compositions, methods, and kits disclosed. In the drawings:

FIG. 1 shows a reaction scheme utilized to yield a self-assembled, biodegradable neuroscaffold formed in situ via copper-free click chemistry-mediated covalent cross-linking of surface-functionalized microparticles, nanoparticles, or any combination thereof, through an azide-alkyne cyclo-addition reaction mechanism.

FIG. 2 shows a reaction scheme utilized to yield a self-assembled, biodegradable neuroscaffold formed in situ via copper-free click chemistry-mediated covalent cross-linking of surface-functionalized microparticles, nanoparticles, or any combination thereof, through a tetrazine-alkene ligation.

FIG. 3 shows a reaction scheme utilized to yield a self-assembled, biodegradable neuroscaffold formed in situ via copper-free click chemistry-mediated covalent cross-linking of surface-functionalized microparticles, nanoparticles, or any combination thereof, and end group functionalized multi-arm poly(ethylene glycol) through a tetrazine-alkene ligation.

FIG. 4 shows the change in the storage modulus with time at constant frequency and constant temperature immediately after tetrazine modified microparticles and nanoparticles and tetrazine modified multi-arm poly(ethylene glycol) are combined with trans-cyclooctene modified microparticles and nanoparticles and trans-cyclooctene modified multi-arm poly(ethylene glycol).

FIG. 5 shows the lack of change in storage modulus and loss modulus with angular frequency at constant temperature during oscillation frequency sweeps after tetrazine-trans-cyclooctene ligation with surface modified microparticles and nanoparticles and end group modified multi-arm poly(ethylene glycol).

FIG. 6 shows a reaction scheme utilized to yield a self-assembled, biodegradable neuroscaffold formed in situ via Michael-type addition-mediated covalent cross-linking of surface-functionalized microparticles, nanoparticles, or any combination thereof.

### DETAILED DESCRIPTION OF ILLUSTRATIVE EMBODIMENTS

The disclosed compositions, methods, and kits may be understood more readily by reference to the following detailed description taken in connection with the accompanying figures, which form a part of this disclosure. It is to be understood that the disclosed compositions, methods, and kits are not limited to the specific compositions, methods, and kits described and/or shown herein, and that the terminology used herein is for the purpose of describing particular embodiments by way of example only and is not intended to be limiting of the claimed compositions, methods, and kits. Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Further, reference to values stated in ranges include each and every value within that range. All ranges are inclusive and combinable. Similarly, when values are expressed as approximations, by use of the antecedent “about,” it will be understood that the particular value forms another embodiment.

It is to be appreciated that certain features of the disclosed compositions, methods, and kits which are, for clarity, described herein in the context of separate embodiments, may also be provided in combination in a single embodiment. Conversely, various features of the disclosed compositions, methods, and kits that are, for brevity, described in the context of a single embodiment, may also be provided separately or in any subcombination.

The term “about” when used in reference to numerical ranges, cutoffs, or specific values is used to indicate that the recited values may vary by up to as much as 25% from the listed value. As many of the numerical values used herein are experimentally determined, it should be understood by those skilled in the art that such determinations can, and often times will, vary among different experiments. The values used herein should not be considered unduly limiting by virtue of this inherent variation. The term “about” is used to encompass variations of ±25% or less, variations of ±20% or less, variations of 10% or less, variations of ±5% or less, variations of ±1% or less, variations of ±0.5% or less, or variations of ±0.1% or less from the specified value.

As used herein, “copper-free click chemistry” means click chemistry performed in the absence of a copper catalyst. It should be understood that those skilled in the art will appreciate the numerous variations of such reaction mechanisms. For example, copper-free click chemistry can follow the synthetic route of an azide-alkyne cyclo-addition reaction mechanism with ring-strained alkyne or a tetrazine-alkene ligation with a ring-strained alkene.

As used herein, “terminal functional group moiety” means a chemically active or reactive group situated at the terminus of a molecule or polymer that is able to participate in a covalent bond or cross-link formation via a copper-free click chemistry or a Michael-type addition reaction mechanism.

As used herein, “capping group moiety” means a chemical group situated at the terminus of a molecule or polymer that is either inert or not able to participate in a covalent bond or cross-link formation via a copper-free click chemistry or a Michael-type addition reaction mechanism.

As used herein, the terms “self-assemble” or “self-assembly” or “self-assembling” mean the ability for microparticles, nanoparticles, polymers, molecules, or any combination thereof, to spontaneously configure themselves via a covalent bond or cross-link formation mechanism to form a larger, defined structure.

As used herein, “spacer or linker moiety” means a homofunctional or heterofunctional molecule or polymer that introduces a defined or controlled space between covalently bonded or cross-linked microparticles, nanoparticles, polymers, molecules, or any combination thereof.

As used herein, “porosity” or “pore size” means either a void space or space containing one or more agents, cells, or any combination thereof. The porosity can range from nanoporous, having pore sizes of at least 1 nanometer and up to 1000 nanometers, to microporous, having pore sizes of up to 500 microns.

As used herein, “exposed on the surface” means that at least a portion of the one or more agents is not covered or encased by the microparticles, nanoparticles, resulting in situ formed neuroscaffold, or any combination thereof, and is accessible from the exterior. The one or more agents exposed on the surface can be fully exposed, such that the entire agent is on the surface of the microparticles, nanoparticles, or resulting in situ formed neuroscaffold, or can be partially exposed, such that only a portion of the agent is on the surface of the microparticles, nanoparticles, or resulting in situ formed neuroscaffold. The one or more agents that are exposed on the surface of the microparticles, nanoparticles, or resulting in situ formed neuroscaffold can be bound to the surface of the biodegradable carrier through, for example, covalent or non-covalent bonds, or can be incorporated within the microparticles, nanoparticles, or resulting in situ formed neuroscaffold, such that a portion of the agent is exposed on the surface.

As used herein, “incorporated within” means that the one or more agents are at least partially covered by, contained within, encased in, or entrapped by the microparticles, nanoparticles, or resulting in situ formed neuroscaffold. In such circumstances, the one or more agents may or may not be exposed on the surface of the microparticles, nanoparticles, or resulting in situ formed neuroscaffold. Depending on the type of microparticles, nanoparticles, or resulting in situ formed neuroscaffold present in the composition, the one or more agents may be located in a void space, such as a core, of the microparticles, nanoparticles, or resulting in situ formed neuroscaffold or dispersed within the microparticles, nanoparticles, or resulting in situ formed neuroscaffold with the potential for being exposed on the surface, or any combination thereof. In some embodiments, the one or more agents can be dispersed or distributed within the microparticles, nanoparticles, or resulting in situ formed neuroscaffold, and not partially exposed on the surface of the biodegradable carrier. In other embodiments, the one or more agents can be partially exposed on the surface of the microparticles, nanoparticles, or resulting in situ formed neuroscaffold. In other embodiments, the one or more agents can be both dispersed or distributed within the microparticles, nanoparticles, or resulting in situ formed neuroscaffold and partially exposed on the surface of the microparticles, nanoparticles, or resulting in situ formed neuroscaffold. In yet other embodiments, the one or more agents can be located in a void space of the microparticles, nanoparticles, or resulting in situ formed neuroscaffold. In yet other embodiments, the one or more agents can be both located in a void space of the microparticles, nanoparticles, or resulting in situ formed neuroscaffold and exposed on the surface of the microparticles, nanoparticles, or resulting in situ formed neuroscaffold.

As used herein, “administering to said subject” and similar terms indicate a procedure by which one or more of the described agents or compositions, together or separately, are introduced into, implanted in, injected into, or applied onto a subject such that target cells, tissues, or segments of the body of the subject are contacted with the agent.

“Pharmaceutically acceptable” refers to those properties and substances which are acceptable to the patient from a pharmacological/toxicological point of view and to the manufacturing pharmaceutical chemist from a physical/chemical point of view regarding composition, formulation, stability, patient acceptance, and bioavailability.

“Pharmaceutically acceptable carrier” refers to a medium that does not interfere with the effectiveness of the biological activity of the active ingredient(s) and is not toxic to the host to which it is administered.

“Therapeutically effective dose” refers to an amount of a composition, as described herein, effective to achieve a particular biological or therapeutic result such as, but not limited to, biological or therapeutic results disclosed, described, or exemplified herein. The therapeutically effective dose may vary according to factors such as the disease state, age, sex, and weight of the individual, and the ability of the composition to cause a desired response in a subject. Such results may include, but are not limited to, the treatment of a spinal cord injury, as determined by any means suitable in the art.

The terms “treating” or “treatment” refer to any success or indicia of success in the attenuation or amelioration of an injury, pathology or condition, including any objective or subjective parameter such as abatement, remission, diminishing of symptoms or making the injury, pathology, or condition more tolerable to the patient, slowing in the rate of inflammation, making the final point of inflammation less debilitating, improving a subject's physical or mental well-being, or prolonging the length of survival. The treatment may be assessed by objective or subjective parameters; including the results of a physical examination, neurological examination, or psychiatric evaluations.

As used herein “focal neurological disorder” means a neurological disorder or disease that is confined or localized to single or punctate neuroanatomical structures or regions in the central or peripheral nervous system, where local therapeutic intervention is achievable. Focal neurological disorders may be caused by or result from nociceptive pain, neuropathic pain, neurotrauma, neuro-inflammation, neurodegenerative diseases, seizure disorders, neurological autoimmune disorders, neuro-oncological diseases, or any combination thereof.

Injectable, In Situ Forming Biodegradable Neuroscaffolds Capable of Providing 3-D Structural Support, Neuroprotection, and/or Subsequent Regeneration in a Subject with a Spinal Cord Injury or a Focal Neurological Disorder.

Disclosed herein are injectable, biodegradable neuroscaffolds formed in situ by self-assembling biodegradable polymeric microparticles, nanoparticles, or any combination thereof, via copper-free click chemistry or Michael-type addition coupling reactions. These injectable, biodegradable neuroscaffolds are designed specifically to provide 3-D structural support, neuroprotection, and/or subsequent regeneration in a subject with a spinal cord injury or a focal neurological disorder.

Injectable, biodegradable neuroscaffolds formed in situ by self-assembling biodegradable polymeric microparticles, nanoparticles, or any combination thereof, via copper-free click chemistry or Michael-type addition coupling reactions offer the ability to overcome the limitations of implantable neuroscaffolds. Injectable, in situ forming biodegradable neuroscaffolds offer the inherent ability to form a 3-D scaffolding matrix that precisely conforms to the neuroanatomical space of interest, dramatically improving the conferred structural support. These injectable, biodegradable neuroscaffolds also enable facile delivery of therapeutically relevant agents and control over release and degradation kinetic profiles. Further, injectable, biodegradable neuroscaffolds that are formed by either copper-free click chemistry or Michael-type addition covalent cross-linking can be formed in the presence of cells, as these coupling chemistries are benign, non-cytotoxic, and yield no reaction by-products. This enables the transplantation of significantly higher percentage and density of viable cells than can be achieved by the cell seeding of implantable, non-injectable neuroscaffolds. This ability to transplant viable cells concurrently with therapeutic agents enables neuroprotection and promotes subsequent tissue regeneration in the disorders of the central and peripheral nervous systems.

Suitable biodegradable polymeric microparticles or nanoparticles can comprise synthetically derived polymers, including, biodegradable polymers and copolymers. Exemplary polymers include, but are not limited to, polyesters, poly(orthoesters), poly(lactides) (PLA), poly(glycolides) (PGA), poly(lactide-co-glycolides) (PLGA), poly(ethylene glycols)(PEG), or any combination thereof. In some embodiments, the synthetically derived biodegradable polymer can be poly(lactic-co-glycolic acid) (PLGA), having a lactic acid and glycolic acid content ranging from 0-100% for each monomer. For example, in some aspects, the biodegradable polymer can be a 50:50 PLGA, where 50:50 refers to the ratio of lactic to glycolic acid. In some embodiments, the biodegradable carrier comprises or consists of a copolymer. For example, in some embodiments, the biodegradable polymer can be a copolymer of poly(ethylene glycol) (PEG) and poly(lactic-co-glycolic acid) (PLGA), having a lactic acid and glycolic acid content ranging from 0-100% for each monomer. Further, in some embodiments, the microparticles and/or nanoparticles can comprise 50:50 PLGA. In other embodiments, the microparticles and/or nanoparticles can comprise a copolymer of 50:50 PLGA and PEG. In yet other embodiments, the microparticles and/or nanoparticles can be cross-linked by suitable terminally functionalized PEGs and/or copolymers of PEG and PLGA.

Further, suitable PEGs include, but are not limited to, linear, branched, multi-armed PEGs having a molecular weight of up to 10,000 g/mol. Additionally, suitable PEGs can be homofunctional or heterofunctional.

Exemplary polymers, including, but not limited to, PEG and copolymers of PLGA and PEG, can further comprise appropriate capping group moieties. Suitable capping group moieties include, but are not limited to, primary amine, carboxyl, hydroxyl, or methoxy.

Further, exemplary polymers, including, but not limited to, PEG and copolymers of PLGA and PEG, can further comprise appropriate terminal functional group moieties capable of undergoing cross-linking via copper-free click chemistry through an azide-alkyne cyclo-addition reaction mechanism. Suitable terminal functional group moieties include, but are not limited to, alkynes, cyclooctynes, substituted cyclooctynes, aryl cyclooctynes, aryl-less cyclooctynes, or azides.

Further, exemplary polymers, including, but not limited to, PEG and copolymers of PLGA and PEG, can further comprise appropriate terminal functional group moieties capable of undergoing cross-linking via copper-free click chemistry via a tetrazine-alkene ligation. Suitable terminal functional group moieties include, but are not limited to, alkenes, trans-cyclooctenes, substituted trans-cyclooctenes, tetrazines, substituted tetrazines, methyltetrazines, or substituted methyltetrazines.

Additionally, exemplary polymers, including, but not limited to, PEG and copolymers of PLGA and PEG, can further comprise appropriate terminal functional group moieties capable of undergoing cross-linking via a Michael-type addition reaction. Suitable terminal functional group moieties include, but are not limited to, alkenes, enones, acrylates, vinyl sulfones, maleimides, or thiols.

Exemplary biodegradable microparticles and/or nanoparticles can be fabricated using processing techniques known by those skilled in the art, including, but not limited to, emulsification, precipitation, or spray drying. In some embodiments, the microparticles and/or nanoparticles can be fabricated by emulsification. In other embodiment, the microparticles and/or nanoparticles can be fabricated by precipitation or nanoprecipitation, respectively. In yet other embodiments, the microparticles and/or nanoparticles can be fabricated by spray drying.

Additionally, the biodegradable microparticles and/or nanoparticles can be fabricated to further comprise one or more agents. Suitable agents include, but are not limited to, small molecules, inhibitors, peptides, proteins, antibodies, growth factors, cytokines, chemokines, neurotrophic factors, oligonucleotides, or any combination thereof. Further, suitable classes of agents include, but are not limited to, analgesics, angiogenesis inhibitors, antibiotics, tetracyclines, anti-anxiety agents, anticonvulsants, antidepressants, tricyclic antidepressants, anti-Parkinsonian agents, antipsychotics, antipsychotropics, anti-inflammatory agents, non-steroidal anti-inflammatory agents, steroids, corticosteroids, anti-arrhythmics, anti-fibrotics, kinase inhibitors, cell cycle inhibitors, cytokine inhibitors, chemokine inhibitors, chemotherapeutics, immunomodulators, immunosuppressants, immunostimulants, cytokines, chemokines, neurotransmitters, neurotrophic factors, neurotrophic agents, neurotrophins, nerve growth factors, or any combination thereof.

Injectable, biodegradable neuroscaffolds can be formed in situ by copper-free click chemistry comprising combining a first suspension of microparticles, nanoparticles, linker moieties, spacer moieties, or any combination thereof, comprising at least two terminal functional alkyne group moieties with a second suspension of microparticles, nanoparticles, linker moieties, spacer moieties, or any combination thereof, comprising at least two terminal functional azide group moieties within a subject, thereby permitting the terminal functional groups of the first suspension to form covalent bonds with the terminal functional groups of the second suspension via a copper-free azide-alkyne cyclo-addition mechanism in order to yield a self-assembled, covalently cross-linked neuroscaffold, provided that at least one of the first suspension or the second suspension comprises microparticles and/or nanoparticles, and wherein the resulting neuroscaffold undergoes hydrolysis or enzymatic cleavage under physiologically relevant conditions. In some embodiments, the terminal functional alkyne group moiety is a cyclooctyne. In some embodiments, the terminal functional alkyne group moiety is a substituted cyclooctyne. In some embodiments, the terminal functional alkyne group moiety is an aryl cyclooctyne. In other embodiments, the terminal functional alkyne group moiety is an aryl-less cyclooctyne.

Injectable, biodegradable neuroscaffolds can be formed in situ by copper-free click chemistry comprising combining a first suspension of microparticles, nanoparticles, linker moieties, spacer moieties, or any combination thereof, comprising at least two terminal functional alkene group moieties with a second suspension of microparticles, nanoparticles, linker moieties, spacer moieties, or any combination thereof, comprising at least two terminal functional tetrazine group moieties within a subject, thereby permitting the terminal functional groups of the first suspension to form covalent bonds with the terminal functional groups of the second suspension via a copper-free tetrazine-alkene ligation in order to yield a self-assembled, covalently cross-linked neuroscaffold, provided that at least one of the first suspension or the second suspension comprises microparticles and/or nanoparticles, and wherein the resulting neuroscaffold undergoes hydrolysis or enzymatic cleavage under physiologically relevant conditions. In some embodiments, the terminal functional alkene group moiety is a trans-cyclooctene. In some embodiments, the terminal functional alkene group moiety is a substituted trans-cyclooctene. In some embodiments, the terminal functional tetrazine group moiety is tetrazine. In some embodiments, the terminal functional tetrazine group moiety is substituted tetrazine. In other embodiments, the terminal functional tetrazine group moiety is a methyltetrazine. In yet other embodiments, the terminal functional tetrazine group moiety is a substituted methyltetrazine.

Injectable, biodegradable neuroscaffolds can be formed in situ by Michael-type addition comprising combining a first suspension of microparticles, nanoparticles, linker moieties, spacer moieties, or any combination thereof, comprising at least two terminal functional alkene group moieties with a second suspension of microparticles, nanoparticles, linker moieties, spacer moieties, or any combination thereof, comprising at least two terminal functional thiol group moieties within a subject, thereby permitting the terminal functional groups of the first suspension to form covalent bonds with the terminal functional groups of the second suspension via a Michael-type addition mechanism in order to yield a self-assembled, covalently cross-linked neuroscaffold, provided that at least one of the first suspension or the second suspension comprises microparticles and/or nanoparticles, and wherein the resulting neuroscaffold undergoes hydrolysis or enzymatic cleavage under physiologically relevant conditions. In some embodiments, the terminal functional alkene group moiety is an acrylate. In other embodiments, the terminal functional alkene group moiety is a vinyl sulfone. In yet other embodiments, the terminal functional alkene group moiety is a maleimide. In yet other embodiments, the terminal functional alkene group moiety is an enone. Further, in some embodiments, the terminal functional thiol group moiety is reduced by a physiologically relevant reducing agent prior to participation in the Michael-type addition cross-linking reaction.

Suitable functionalized spacer or linker moieties include, but are not limited to, a diol, a tetraglycol, a linear PEG, a multi-arm PEG, a branched PEG, a copolymer of PLGA and PEG, a copolymer of PLA and PEG, a copolymer of PGA and PEG, or any combination thereof, comprising at least two terminal functional group moieties capable of undergoing covalent cross-linking reaction via copper-free click chemistry or Michael-type addition.

Additionally, the injectable, biodegradable neuroscaffold formed in situ can be formed in the presence of one or more agents and/or cells. Suitable agents include, but are not limited to, small molecules, inhibitors, peptides, proteins, antibodies, growth factors, cytokines, chemokines, neurotrophic factors, oligonucleotides, or any combination thereof. Suitable cells include, but are not limited to, stem cells, immune cells, neuronal cells, or any combination thereof.

The mechanical properties and/or porosity of the injectable, biodegradable neuroscaffold formed in situ can be controlled by manipulating the concentration and size distribution of the first suspension of microparticles and/or nanoparticles, the second suspension of microparticles and/or nanoparticles, or any combination thereof. For example, increasing the concentration or particle density of nanoparticles will yield a neuroscaffold with a higher cross-link density, thus resulting in a lower porosity and a decreased degradation rate. Additionally, the mechanical properties and/or porosity of the injectable, biodegradable neuroscaffold formed in situ can be further controlled by the addition of linker or spacer moieties, comprising at least two appropriate, cross-linkable terminal functional groups, to the first and/or second suspension of microparticles and/or nanoparticles. For example, an increase in the concentration of spacer or linker moieties provided in the first and/or second suspensions will alter the mechanical properties of the neuroscaffold by lowering the elastic modulus and increasing the porosity and degradation rate. In some embodiments, the mechanical properties can be designed to match the mechanical properties of the surrounding tissues. In other embodiments, the porosity can range from nanoporous, having pore sizes of at least one nanometer and up to 1000 nanometers, to microporous, having pore sizes of up to 500 microns. In yet other embodiments, the porosity can be additionally defined by the incorporation of cells. For example, the porosity and/or pore size of the neuroscaffold formed in situ around or in the presence of cells can be increased by increasing the density of cells to be incorporated.

The injectable, biodegradable neuroscaffold formed in situ can be designed to begin to degrade within any suitable time frame following administration to a subject. In some embodiments, the injectable, biodegradable neuroscaffold formed in situ can begin to degrade from the time of being administered to about 2 years following being administered to a subject. In other embodiments, degradation of 50% of the in situ formed neuroscaffold occurs between the time of formation and about 1 hour, 2 hours, 3 hours, 4 hours, 6 hours, 8 hours, 10 hours, 12 hours, 16 hours, 20 hours, 1 day, 2 days, 3 days, 4 days, 5 days, 6 days, 7 days, 8 days, 9 days, 10 days, 12 days, 14 days, 16 days, 18 days, 21 days, 24 days, 28 days, 35 days, 42 days, 49 days, 2 months, 3 months, 4 months, 5 months, 6 months, 7 months, 8 months, 9 months, 10 months, 11 months, 12 months, 14 months, 16 months, 18 months, 21 months, or 24 months, inclusive, post-formation. In yet other embodiments, the degradation of the neuroscaffold formed in situ leaves no residual in the site of administration.

The injectable, biodegradable neuroscaffold formed in situ can be designed to release one or more agents for any desired period of time as a result of degradation, diffusion, or any combination thereof. In some embodiments, the injectable, biodegradable neuroscaffold formed in situ can be designed to release less than 60% of one or more agents between the time of injection and about 1 hour, 2 hours, 3 hours, 4 hours, 6 hours, 8 hours, 10 hours, 12 hours, 16 hours, 20 hours, 1 day, 2 days, 3 days, 4 days, 5 days, 6 days, 7 days, 8 days, 9 days, 10 days, 12 days, 14 days, 16 days, 18 days, 21 days, 24 days, 28 days, 35 days, 42 days, 49 days, 2 months, 3 months, 4 months, 5 months, 6 months, 7 months, 8 months, 9 months, 10 months, 11 months, 12 months, 14 months, 16 months, 18 months, 21 months, or 24 months, inclusive, post-injection.

The release of one or more agents from the injectable, biodegradable neuroscaffold formed in situ can result in therapeutic efficacy. In some embodiments, release of one or more agents from the injectable, biodegradable neuroscaffold formed in situ can provide a therapeutically efficacious dose of an agent from the time of injection to about 1 hour, 2 hours, 3 hours, 4 hours, 6 hours, 8 hours, 10 hours, 12 hours, 16 hours, 20 hours, 1 day, 2 days, 3 days, 4 days, 5 days, 6 days, 7 days, 8 days, 9 days, 10 days, 12 days, 14 days, 16 days, 18 days, 21 days, 24 days, 28 days, 35 days, 42 days, 49 days, 2 months, 3 months, 4 months, 5 months, 6 months, 7 months, 8 months, 9 months, 10 months, 11 months, 12 months, 14 months, 16 months, 18 months, 21 months, or 24 months, inclusive, post-injection.

The components that are used to form the injectable, biodegradable neuroscaffold (e.g., the first suspension or the second suspension) can further comprise a pharmaceutically acceptable carrier or excipient, as would be known to an individual skilled in the relevant art.

The described injectable, biodegradable neuroscaffold compositions may be formulated as any of various preparations that are known and suitable in the art, including those described and exemplified herein. In some embodiments, the injectable, biodegradable neuroscaffold compositions are initially aqueous formulations and/or suspensions. Aqueous formulations, solutions, and/or suspensions may be prepared by admixing the described compositions in water or suitable physiologic buffer, and optionally adding suitable colorants, preservatives, stabilizing and thickening agents, ions such as calcium or magnesium, and the like as desired. Aqueous formulations and/or suspensions may also be made by dispersing the described injectable, biodegradable neuroscaffold compositions in water or physiologic buffer with viscous material, such as natural or synthetic gums, resins, methylcellulose, sodium carboxymethylcellulose, and other well-known suspending agents. Also included are liquid formulations and solid form preparations which are intended to be converted, shortly before use, to liquid preparations. Such liquids include solutions, suspensions, syrups, slurries, and emulsions. Liquid preparations may be prepared by conventional means with pharmaceutically acceptable additives such as suspending agents (e.g., sorbitol syrup, cellulose derivatives or hydrogenated edible fats or oils); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). These preparations may contain, in addition to the active agent, stabilizers, buffers, dispersants, thickeners, solubilizing agents, and the like. The injectable, biodegradable neuroscaffold compositions may be in powder or lyophilized form for constitution with a suitable vehicle such as sterile water, physiological buffer, or saline solution before use. The injectable, biodegradable neuroscaffold compositions may be formulated for injection into a subject. For injection, the injectable, biodegradable neuroscaffold compositions described may be formulated in aqueous solutions such as water, or in physiologically compatible buffers such as Hanks's solution, Ringer's solution, physiological saline buffer, or artificial cerebral spinal fluid. The solution may contain one or more formulatory agents such as suspending, stabilizing or dispersing agents. Injection formulations may also be prepared as solid form preparations which are intended to be converted, shortly before use, to liquid form preparations suitable for injection, for example, by constitution with a suitable vehicle, such as sterile water, saline solution, or artificial cerebral spinal fluid before use.

The disclosed injectable, biodegradable neuroscaffold can be administered to provide 3-dimensional (3-D) structural support, neuroprotection and/or subsequent regeneration in a subject with a spinal cord injury or a focal neurological disorder.

Focal neurological disorders may be caused by or result from nociceptive pain, neuropathic pain, neurotrauma, neuro-inflammation, neurodegenerative diseases, seizure disorders, neurological autoimmune disorders, neuro-oncological diseases, or any combination thereof

For providing 3-dimensional (3-D) structural support, neuroprotection and/or subsequent regeneration in a subject with a spinal cord injury, the disclosed injectable, biodegradable neuroscaffold can be administered to the spinal cord of the subject. For example, the injectable, biodegradable neuroscaffold can be administered by direct injection into, near, around, or within close proximity of the spinal cord of the subject.

As the injuries suitable for treatment include traumatic bodily injuries that affect the spinal cord, the described methods may be carried out when the temperature of the body or spinal region has been lowered. In some embodiments the described injectable, biodegradable neuroscaffold compositions may be administered when the spinal cord of the subject is from about 96° F. to about 85° F. In some embodiments the described injectable, biodegradable neuroscaffold compositions may be administered when the spinal cord of the subject is about 96° F., about 95° F., about 94° F., about 93° F., about 92° F., about 91° F., about 90° F., about 89° F., about 88° F., or about 87° F.

Also, because rapid treatment is often desirable for spinal cord injuries, the described methods may be carried out within about 2 hours of a subject's spinal cord injury. In some embodiments the described methods may be carried out within about 4 hours of a subject's spinal cord injury. In some embodiments the described methods may be carried out within about 6 hours of a subject's spinal cord injury. In some embodiments the described methods may be carried out within about 12 hours of a subject's spinal cord injury. In some embodiments the described methods may be carried out within about 18 hours of a subject's spinal cord injury. In some embodiments the described methods may be carried out within about 24 hours of a subject's spinal cord injury. In some embodiments the described methods may be carried out within about 36 hours of a subject's spinal cord injury. In some embodiments the described methods may be carried out within about 48 hours of a subject's spinal cord injury. In some embodiments the described methods may be carried out within about 72 hours of a subject's spinal cord injury. In some embodiments, the described methods can be carried out from the time of a subject's spinal cord injury to about 1 week after a subject's spinal cord injury. In other embodiments, the described methods can be carried out from the time of a subject's spinal cord injury to about 72 hours after a subject's spinal cord injury. In other embodiments, the described methods can be carried out from the time of a subject's spinal cord injury to about 48 hours after a subject's spinal cord injury. In other embodiments, the described methods can be carried out from the time of a subject's spinal cord injury to about 24 hours after a subject's spinal cord injury. In some embodiments, the described methods can be carried out from about 24 hours after a subject's spinal cord injury to about 1 week after a subject's spinal cord injury. In other embodiments, the described methods can be carried out from about 24 hours after a subject's spinal cord injury to about 72 hours after a subject's spinal cord injury. In other embodiments, the described methods can be carried out from about 24 hours after a subject's spinal cord injury to about 48 hours after a subject's spinal cord injury. In some embodiments, the described methods can be carried out from about 48 hours after a subject's spinal cord injury to about 1 week after a subject's spinal cord injury. In other embodiments, the described methods can be carried out from about 48 hours after a subject's spinal cord injury to about 72 hours after a subject's spinal cord injury.

In some embodiments the described methods may be carried out within about 1 week of initiation of treatment for a subject's spinal cord injury. In some embodiments the described methods may be carried out within about 72 hours of initiation of treatment for a subject's spinal cord injury. In some embodiments the described methods may be carried out within about 48 hours of initiation of treatment for a subject's spinal cord injury. In some embodiments the described methods may be carried out within about 24 hours of initiation of treatment for a subject's spinal cord injury. In some embodiments the described methods may be carried out within about 18 hours of initiation of treatment for a subject's spinal cord injury. In some embodiments the described methods may be carried out within about 12 hours of initiation of treatment for a subject's spinal cord injury. In some embodiments the described methods may be carried out within about 6 hours of initiation of treatment for a subject's spinal cord injury. In some embodiments the described methods may be carried out within about 4 hours of initiation of treatment for a subject's spinal cord injury. In some embodiments the described methods may be carried out within about 3 hours of initiation of treatment for a subject's spinal cord injury. In some embodiments the described methods may be carried out within about 2 hours of initiation of treatment for a subject's spinal cord injury. In some embodiments the described methods may be carried out within about 1 hour of initiation of treatment for a subject's spinal cord injury. In some embodiments the described methods may be carried out less than 1 hour after initiation of treatment for a subject's spinal cord injury.

For providing 3-dimensional (3-D) structural support, neuroprotection and/or subsequent regeneration in a subject with a focal neurological disorder, the disclosed injectable, biodegradable neuroscaffold can be administered to circumvent the blood-brain barrier (BBB). For example, the injectable, biodegradable neuroscaffold can be administered by direct injection into, near, around, or within close proximity to the focal lesion electrophysiologically identified in a subject having seizure disorders. In some embodiments, the injectable, biodegradable neuroscaffold can be administered at the site of tissue or tumor resection. In other embodiments, the injectable, biodegradable neuroscaffold can be administered by direct injection into, near, around, or within close proximity to a neuroanatomical structure known to cause neurodegenerative disorders. For example, the injectable, biodegradable neuroscaffold can be inject near, around or within close proximity to the substantia nigra.

In some embodiments the described methods may be carried out within any desired or suitable time following the diagnosis of the focal neurological disorder. In other embodiments, the described methods may be carried out with any desired or suitable time following the initiation of treatment for a subject's focal neurological disorder.

Also provided herein are kits for producing a composition for an injectable, in situ forming biodegradable neuroscaffold capable of providing 3-D structural support and subsequent neuroprotection in a subject with a spinal cord injury; and instructions for producing said composition.

### EXAMPLES

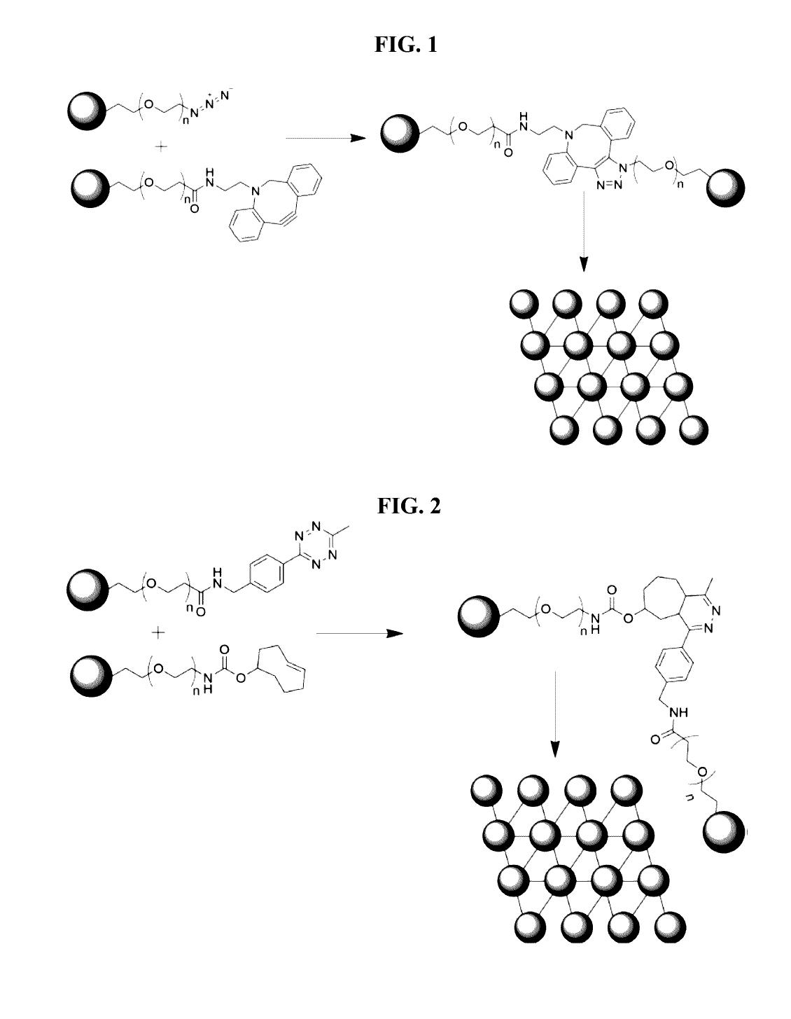

Preparation of an injectable, in situ forming neuroscaffold through covalent self-assembly of PLGA-g-PEG nanoparticles via copper free click chemistry through an azide-alkyne cyclo-addition mechanism. Varying ratios of PLGA-g-PEG and PLGA-g-PEG-azide or PLGA-g-PEG and PLGA-g-PEG-dibenzylcyclooctyne diblock copolymer (0-5% by weight) are dissolved in a water miscible solvent (e.g. acetonitrile, dimethylsulfoxide, N,N-dimethylformamide, acetone, or solvent mixtures). The polymer solution is precipitated into water, a nonsolvent, to yield a nanoparticles comprising a PEGylated surface with varying percentages of PEG-azide or PEG-dibenzylcyclooctyne functionality (0-50 mole percent). The resulting nanoparticle suspension is stirred for 2-6 hours enable sufficient solvent diffusion. The nanoparticle suspension is then purified and concentrated by ultrafiltration and lyophilized. Azide-functionalized nanoparticles and dibenzylcyclooctyne-functionalized nanoparticles (1:1 stoichiometric ratio of terminal azide to terminal dibenzylcyclooctyne functional groups) are resuspended independently in buffered saline (pH 7.4) for injection and subsequently mixed in situ to covalently crosslink the nanoparticle surface via copper-free click chemistry, resulting in an in situ formed neuroscaffold (FIG. 1).

Preparation of an injectable, in situ forming neuroscaffold through covalent self-assembly of PLGA-g-PEG nanoparticles via copper free click chemistry through a tetrazine-alkene ligation. Varying ratios of PLGA-g-PEG and PLGA-g-PEG-methyltetrazine or PLGA-g-PEG and PLGA-g-PEG-trans-cyclooctene diblock copolymer (0-5% by weight) are dissolved in a water miscible solvent (e.g. acetonitrile, dimethylsulfoxide, N,N-dimethylformamide, acetone, or solvent mixtures). The polymer solution is precipitated into water, a nonsolvent, to yield a nanoparticles comprising a PEGylated surface with varying percentages of PEG-methyltetrazine or PEG-trans-cyclooctene functionality (0-50 mole percent). The resulting nanoparticle suspension is stirred for 2-6 hours enable sufficient solvent diffusion. The nanoparticle suspension is then purified and concentrated by ultrafiltration and lyophilized. Methyltetrazine-functionalized nanoparticles and trans-cyclooctene-functionalized nanoparticles (1:1 stoichiometric ratio of terminal methyltetrazine to terminal trans-cyclooctene functional groups) are resuspended independently in buffered saline (pH 7.4) for injection and subsequently mixed in situ to covalently crosslink the nanoparticle surface via copper-free click chemistry, resulting in an in situ formed neuroscaffold (FIG. 2).

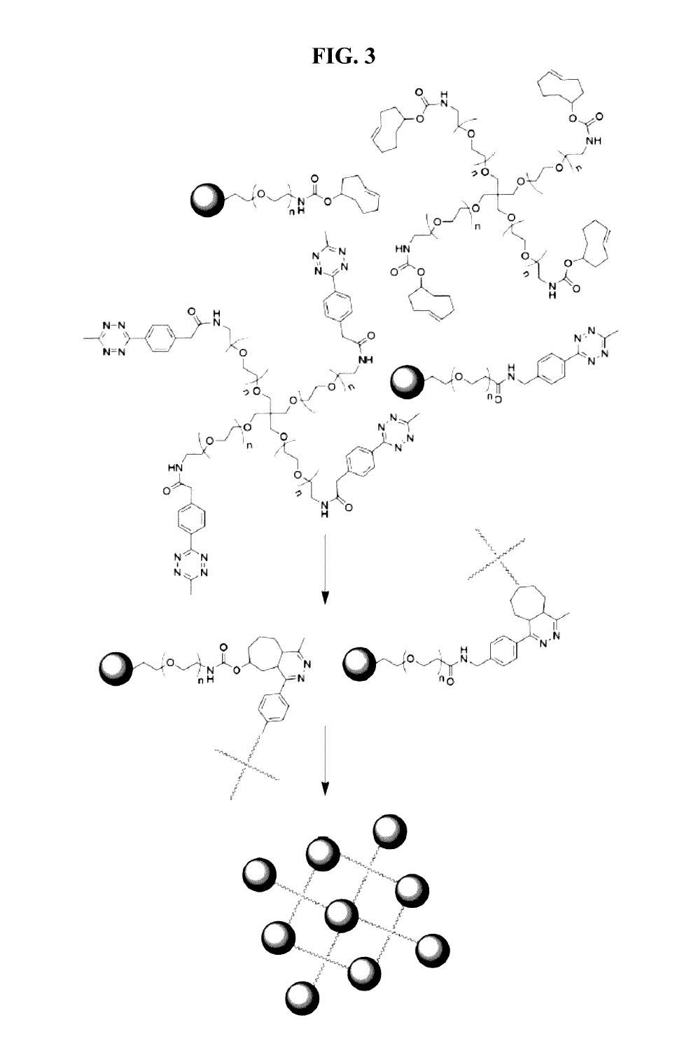

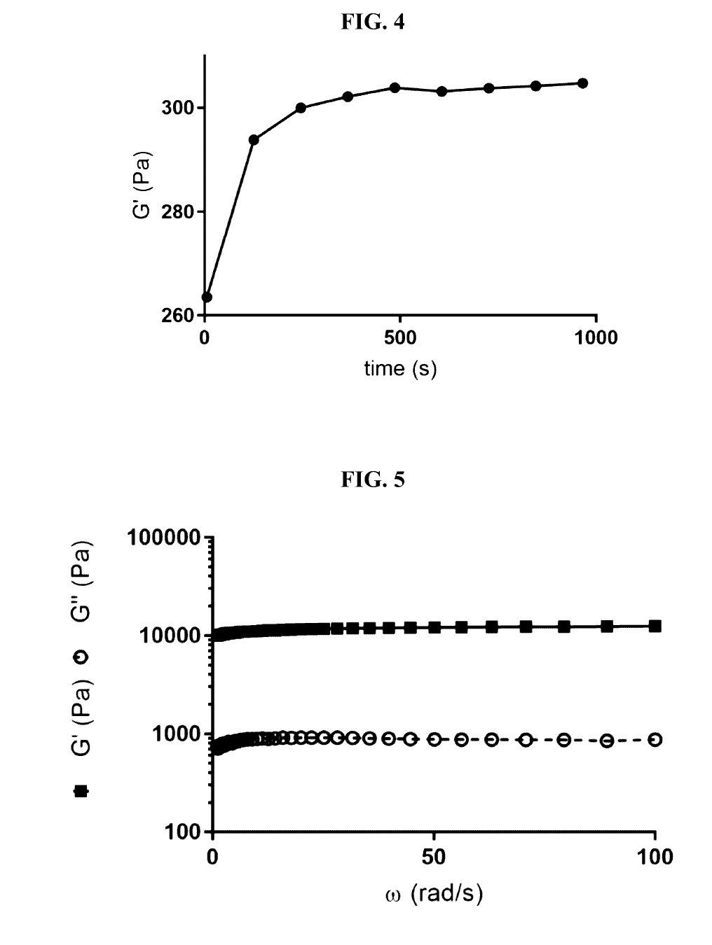

Preparation of an injectable, in situ forming neuroscaffold through covalent self-assembly of PLGA-g-PEG nanoparticles and multi-arm PEGs via copper free click chemistry through a tetrazine-alkene ligation. Varying ratios of PLGA-g-PEG and PLGA-g-PEG-methyltetrazine or PLGA-g-PEG and PLGA-g-PEG-trans-cyclooctene diblock copolymer (0-5% by weight) are dissolved in a water miscible solvent (e.g. acetonitrile, dimethylsulfoxide, N,N-dimethylformamide, acetone, or solvent mixtures). The polymer solution is precipitated into water, a nonsolvent, to yield a nanoparticles comprising a PEGylated surface with varying percentages of PEG-methyltetrazine or PEG-trans-cyclooctene functionality (0-50 mole percent). The resulting nanoparticle suspension is stirred for 2-6 hours enable sufficient solvent diffusion. The nanoparticle suspension is then purified and concentrated by ultrafiltration and lyophilized. Methyltetrazine-functionalized nanoparticles, methyltetrazine-functionalized multi-arm PEG, trans-cyclooctene-functionalized nanoparticles, and trans-cyclooctene-functionalized multi-arm PEG (1:1 stoichiometric ratio of terminal methyltetrazine to terminal trans-cyclooctene functional groups) are resuspended independently by functional group in buffered saline (pH 7.4) for injection and subsequently mixed in situ to covalently crosslink the nanoparticle surface via copper-free click chemistry, resulting in an in situ formed neuroscaffold (FIGS. 3-5).

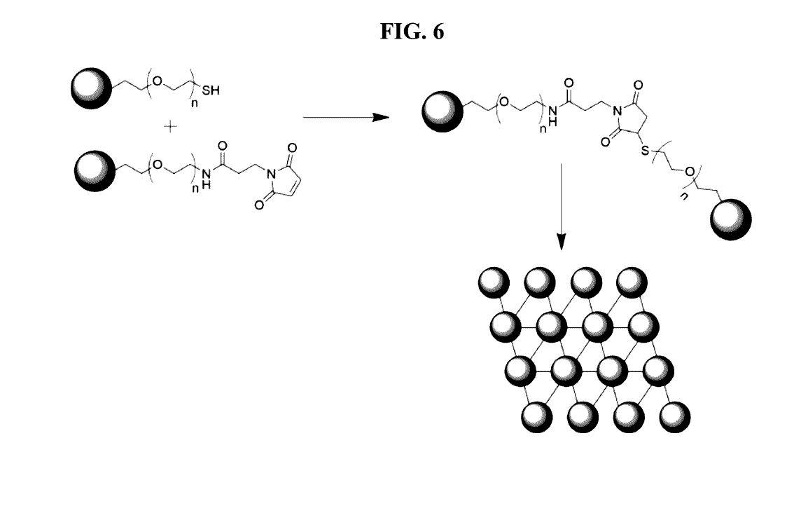

Preparation of an injectable, in situ forming neuroscaffold through covalent self-assembly of PLGA-g-PEG nanoparticles via Michael-type addition. Varying ratios of PLGA-g-PEG and PLGA-g-PEG-thiol or PLGA-g-PEG and PLGA-g-PEG-maleimide diblock copolymer (0-5% by weight) are dissolved in a water miscible solvent (e.g. acetonitrile, dimethylsulfoxide, N,N-dimethylformamide, acetone, or solvent mixtures). The polymer solution is precipitated into water, a nonsolvent, to yield a nanoparticles comprising a PEGylated surface with varying percentages of PEG-thiol or PEG-maleimide functionality (0-50 mole percent). The resulting nanoparticle suspension is stirred for 2-6 hours enable sufficient solvent diffusion. The nanoparticle suspension is then purified and concentrated by ultrafiltration and lyophilized. Thiol-functional nanoparticles are resuspended in buffered saline (pH 7.4) containing reduced glutathione and maleimide-functionalized nanoparticles (1:1 stoichiometric ratio of terminal thiol to terminal maleimide functional groups) are resuspended independently in buffered saline (pH 7.4) for injection and subsequently mixed in situ to covalently crosslink the nanoparticle surface via a Michael-type addition reaction, resulting in an in situ formed neuroscaffold (FIG. 6).

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed:

1. An injectable, biodegradable neuroscaffold formed in situ by the self-assembly of surface-functionalized biodegradable, polymeric microparticles, nanoparticles, or any combination thereof comprising at least one terminal functional group moiety that is capable of undergoing a covalent cross-linking reaction with at least one other terminal functional group moiety of said microparticles, nanoparticles, or a combination thereof via
copper-free click chemistry; or,
Michael-type addition,
wherein the resulting neuroscaffold comprises at least one reducible, hydrolytically cleavable, or enzymatically cleavable bond under physiologically relevant conditions; and,
wherein the resulting neuroscaffold comprises mechanical properties that are controlled by performing the cross-linking in the presence or absence of a functionalized linker or spacer moiety comprising at least two terminal functional group moieties capable of undergoing said covalent cross-linking reactions.

2. The injectable, biodegradable neuroscaffold according to claim 1, wherein the biodegradable, polymeric microparticles, nanoparticles, or a combination thereof, comprise poly(lactide-co-glycolides) (PLGA), poly(lactides) (PLA), poly(glycolides) (PGA), copolymers of PLGA, PLA, or PGA and a poly(ethylene glycol) (PEG) having a molecular weight of up to 10,000 g/mol, or any combination thereof.

3. The injectable, biodegradable neuroscaffold according to claim 2, wherein the PEG copolymer comprises either a terminal functional group moiety capable of undergoing a covalent cross-linking reaction via copper-free click chemistry or Michael-type addition or a capping group.

4. The injectable, biodegradable neuroscaffold according to claim 3, wherein the capping group comprises a primary amine, a carboxyl, a hydroxyl, or a methoxy.

5. The injectable, biodegradable neuroscaffold according to claim 1, wherein the terminal functional group moiety capable of undergoing covalent cross-linking via copper-free click chemistry comprises a cyclooctyne, a substituted cyclooctyne, an aryl cyclooctyne, an aryl-less cyclooctyne, or an azide.

6. The injectable, biodegradable neuroscaffold according to claim 1, wherein the terminal functional group moiety capable of undergoing covalent cross-linking via copper-free click chemistry comprises a trans-cyclooctene, a substituted trans-cyclooctene, an alkene, a tetrazine, a substituted tetrazine, a methyltetrazine, or a substituted methyltetrazine.

7. The injectable, biodegradable neuroscaffold according to claim 1, wherein the terminal functional group moiety capable of undergoing covalent cross-linking via Michael-type addition comprises an alkene, an enone, a vinyl sulfone, a maleimide or a thiol.

8. The injectable, biodegradable neuroscaffold according to claim 1, wherein the covalent cross-linking reaction via Michael-type addition is performed in the presence of a physiologically relevant reducing agent.

9. The injectable, biodegradable neuroscaffold according to claim 8, wherein the physiologically relevant reducing agent is glutathione.

10. The injectable, biodegradable neuroscaffold according to claim 1, wherein the functionalized linker or spacer moiety is a diol, a tetraglycol, a linear PEG, a multi-arm PEG, a branched PEG, a copolymer of PLGA and PEG, a copolymer of PLA and PEG, a copolymer of PGA and PEG, or any combination thereof, comprising at least two terminal functional group moieties capable of undergoing covalent cross-linking reaction via copper-free click chemistry or Michael-type addition.

11. The injectable, biodegradable neuroscaffold according to claim 10, wherein the PEG has a molecular weight of up to 10,000 g/mol.

12. The injectable, biodegradable neuroscaffold according to claim 10, wherein the terminal functional group moieties capable of undergoing a covalent cross-linking reaction via copper-free click chemistry are cyclooctynes, substituted cyclooctynes, aryl cyclooctynes, aryl-less cyclooctynes, azides, or any combination thereof.

13. The injectable, biodegradable neuroscaffold according to claim 10, wherein the terminal functional group moieties capable of undergoing a covalent cross-linking reaction via copper-free click chemistry are trans-cyclooctenes, substituted trans-cyclooctenes, alkenes, tetrazines, substituted tetrazines, methyltetrazines, substituted methyltetrazines. or any combination thereof.

14. The injectable, biodegradable neuroscaffold according to claim 10, wherein the terminal functional group moieties capable of undergoing a covalent cross-linking reaction via Michael-type addition are alkenes, enones, acrylates, vinyl sulfones, maleimides, thiols, or any combination thereof.

15. The injectable, biodegradable neuroscaffold according to claim 1, wherein the microparticles, nanoparticles, or a combination thereof, are fabricated by emulsification, precipitation, nanoprecipitation, spray drying, or any combination thereof.

16. The injectable, biodegradable neuroscaffold according to claim 1, wherein the microparticles, nanoparticles, or a combination thereof, further comprise one or more agents.

17. The injectable, biodegradable neuroscaffold according to claim 16, wherein the one or more agents is a small-molecule, an inhibitor, a peptide, a protein, an antibody, a growth factor, a cytokine, a chemokine, a neurotrophic factor, an oligonucleotide, or any combination thereof.

18. The injectable, biodegradable neuroscaffold according to claim 16, wherein the one or more agents is incorporated within the microparticles or nanoparticles, exposed on the surface of the microparticles or nanoparticles, or a combination thereof.

19. The injectable, biodegradable neuroscaffold according to claim 16, wherein the one or more agents is incorporated within the neuroscaffold formed in situ, exposed on the surface of the neuroscaffold formed in situ, or any combination thereof.

20. The injectable, biodegradable neuroscaffold according to claim 1, wherein the in situ self-assembly is performed in the presence of one or more agents, transplantable cells, or any combination thereof.

21. The injectable, biodegradable neuroscaffold according to claim 1, wherein the porosity ranges from nanoporous, having pore sizes of at least 1 nanometer and up to 1000 nanometers, to microporous, having pore sizes of up to 500 microns.

22. The injectable, biodegradable neuroscaffold according to claim 1, wherein the mechanical properties are selected and controlled according to the neuroanatomical tissue of interest.

23. The injectable, biodegradable neuroscaffold according to claim 1, wherein biodegradation of 50% of the in situ formed neuroscaffold occurs between the time of formation and about 1 hour, 2 hours, 3 hours, 4 hours, 6 hours, 8 hours, 10 hours, 12 hours, 16 hours, 20 hours, 1 day, 2 days, 3 days, 4 days, 5 days, 6 days, 7 days, 8 days, 9 days, 10 days, 12 days, 14 days, 16 days, 18 days, 21 days, 24 days, 28 days, 35 days, 42 days, 49 days, 2 months, 3 months, 4 months, 5 months, 6 months, 7 months, 8 months, 9 months, 10 months, 11 months, 12 months, 14 months, 16 months, 18 months, 21 months, or 24 months, inclusive, post-formation.

24. The injectable, biodegradable neuroscaffold according to claim 1, wherein the in situ formed neuroscaffold releases less than 60% of the one or more agents between the time of injection and about 1 hour, 2 hours, 3 hours, 4 hours, 6 hours, 8 hours, 10 hours, 12 hours, 16 hours, 20 hours, 1 day, 2 days, 3 days, 4 days, 5 days, 6 days, 7 days, 8 days, 9 days, 10 days, 12 days, 14 days, 16 days, 18 days, 21 days, 24 days, 28 days, 35 days, 42 days, 49 days, 2 months, 3 months, 4 months, 5 months, 6 months, 7 months, 8 months, 9 months, 10 months, 11 months, 12 months, 14 months, 16 months, 18 months, 21 months, or 24 months, inclusive, post-injection.

25. The injectable, biodegradable neuroscaffold according to claim 1, wherein the in situ formed neuroscaffold provides a therapeutically efficacious dose of one or more agents from the time of injection to about 1 hour, 2 hours, 3 hours, 4 hours, 6 hours, 8 hours, 10 hours, 12 hours, 16 hours, 20 hours, 1 day, 2 days, 3 days, 4 days, 5 days, 6 days, 7 days, 8 days, 9 days, 10 days, 12 days, 14 days, 16 days, 18 days, 21 days, 24 days, 28 days, 35 days, 42 days, 49 days, 2 months, 3 months, 4 months, 5 months, 6 months, 7 months, 8 months, 9 months, 10 months, 11 months, 12 months, 14 months, 16 months, 18 months, 21 months, or 24 months, inclusive, post-injection.

26. The components used to form the injectable, biodegradable neuroscaffold according to claim 1, further comprising a pharmaceutically acceptable carrier or excipient.

27. A method for forming an injectable, biodegradable neuroscaffold in situ via copper-free click chemistry comprising:
combining a first suspension of microparticles, nanoparticles, linker moieties, spacer moieties, or any combination thereof, comprising at least two terminal functional alkyne group moieties with a second suspension of microparticles, nanoparticles, linker moieties, spacer moieties, or any combination thereof, comprising at least two terminal functional azide group moieties within a subject, thereby permitting the terminal functional groups of the first suspension to form covalent bonds with the terminal functional groups of the second suspension via a copper-free azide-alkyne cyclo-addition mechanism or a copper-free tetrazine-alkene ligation in order to yield a self-assembled, covalently cross-linked neuroscaffold with controllable mechanical properties,
provided that at least one of the first suspension or the second suspension comprises microparticles, nanoparticles, or a combination thereof,
wherein the resulting neuroscaffold comprises at least one reducible, hydrolytically cleavable, or enzymatically cleavable bond under physiologically relevant conditions.

28. The method of claim 27, wherein the biodegradable, polymeric microparticles, nanoparticles, or a combination thereof, comprise poly(lactide-co-glycolides) (PLGA), poly(lactides) (PLA), poly(glycolides) (PGA), copolymers of PLGA, PLA, or PGA and a poly(ethylene glycol) (PEG) having a molecular weight of up to 10,000 g/mol, or any combination thereof.

29. The method of claim 28, wherein the PEG copolymer comprises either a terminal functional group moiety that is capable of undergoing a covalent cross-linking reaction via copper-free click chemistry or a capping group.

30. The method of claim 28, wherein the capping group comprises a primary amine, a carboxyl, a hydroxyl, or a methoxy.

31. The method of claim 27, wherein the terminal functional group moiety capable of undergoing covalent cross-linking via copper-free click chemistry comprises a cyclooctyne, a substituted cyclooctyne, an aryl cyclooctyne, an aryl-less cyclooctyne, or an azide.

32. The method of claim 27, wherein the terminal functional group moiety capable of undergoing covalent cross-linking via copper-free click chemistry comprises a trans-cyclooctene, a substituted trans-cyclooctene, an alkene, a tetrazine, a substituted tetrazine, a methyltetrazine, or a substituted methyltetrazine.

33. The method of claim 27, wherein the functionalized linker or spacer moiety is a linear PEG, a multi-arm PEG, a branched PEG, a copolymer of PLGA and PEG, a copolymer of PLA and PEG, a copolymer of PGA and PEG, or any combination thereof, comprising at least two terminal functional group moieties capable of undergoing covalent cross-linking reaction via copper-free click chemistry.

34. The method of claim 33, wherein the PEG has a molecular weight of up to 10,000 g/mol.

35. The method of claim 33, wherein the terminal functional group moieties capable of undergoing a covalent cross-linking reaction via copper-free click chemistry are cyclooctynes, substituted cyclooctynes, aryl cyclooctynes, aryl-less cyclooctynes, azides, or any combination thereof.

36. The method of claim 33, wherein the terminal functional group moieties capable of undergoing a covalent cross-linking reaction via copper-free click chemistry are trans-cyclooctenes, substituted trans-cyclooctenes, alkenes, tetrazines, substituted tetrazines, methyltetrazines, substituted methyltetrazines. or any combination thereof.

37. The method of claim 27, wherein the microparticles, nanoparticles, or a combination thereof, are fabricated by emulsification, precipitation, nanoprecipitation, spray drying, or any combination thereof.

38. The method of claim 27, wherein the microparticles, nanoparticles, or a combination thereof, further comprise one or more agents.

39. The method of claim 38, wherein the one or more agents is a small-molecule, an inhibitor, a peptide, a protein, an antibody, a growth factor, a cytokine, a chemokine, a neurotrophic factor, an oligonucleotide, or any combination thereof.

40. The method of claim 38, wherein the one or more agents is incorporated within the microparticles or nanoparticles, exposed on the surface of the microparticles or nanoparticles, or any combination thereof.

41. The method of claim 38, wherein the one or more agents is incorporated within the neuroscaffold formed in situ, exposed on the surface of the neuroscaffold formed in situ, or any combination thereof.

42. The method of claim 27, wherein the in situ self-assembly is performed in the presence of one or more agents and/or transplantable cells.

43. The method of claim 27, wherein the porosity ranges from nanoporous, having pore sizes of at least 1 nanometer and up to 1000 nanometers, to microporous, having pore sizes of up to 500 microns.

44. The method of claim 27, wherein the mechanical properties are selected and controlled according to the neuroanatomical tissue of interest.

45. The method of claim 27, wherein biodegradation of 50% of the in situ formed neuroscaffold occurs between the time of formation and about 1 hour, 2 hours, 3 hours, 4 hours, 6 hours, 8 hours, 10 hours, 12 hours, 16 hours, 20 hours, 1 day, 2 days, 3 days, 4 days, 5 days, 6 days, 7 days, 8 days, 9 days, 10 days, 12 days, 14 days, 16 days, 18 days, 21 days, 24 days, 28 days, 35 days, 42 days, 49 days, 2 months, 3 months, 4 months, 5 months, 6 months, 7 months, 8 months, 9 months, 10 months, 11 months, 12 months, 14 months, 16 months, 18 months, 21 months, or 24 months, inclusive, post-formation.

46. The method of claim 27, wherein the in situ formed neuroscaffold releases less than 60% of the one or more agents between the time of injection and about 1 hour, 2 hours, 3 hours, 4 hours, 6 hours, 8 hours, 10 hours, 12 hours, 16 hours, 20 hours, 1 day, 2 days, 3 days, 4 days, 5 days, 6 days, 7 days, 8 days, 9 days, 10 days, 12 days, 14 days, 16 days, 18 days, 21 days, 24 days, 28 days, 35 days, 42 days, 49 days, 2 months, 3 months, 4 months, 5 months, 6 months, 7 months, 8 months, 9 months, 10 months, 11 months, 12 months, 14 months, 16 months, 18 months, 21 months, or 24 months, inclusive, post-injection.

47. The method of claim 27, wherein the in situ formed neuroscaffold provides a therapeutically efficacious dose of one or more agents from the time of injection to about 1 hour, 2 hours, 3 hours, 4 hours, 6 hours, 8 hours, 10 hours, 12 hours, 16 hours, 20 hours, 1 day, 2 days, 3 days, 4 days, 5 days, 6 days, 7 days, 8 days, 9 days, 10 days, 12 days, 14 days, 16 days, 18 days, 21 days, 24 days, 28 days, 35 days, 42 days, 49 days, 2 months, 3 months, 4 months, 5 months, 6 months, 7 months, 8 months, 9 months, 10 months, 11 months, 12 months, 14 months, 16 months, 18 months, 21 months, or 24 months, inclusive, post-injection.

48. The method of claim 27, further comprising a pharmaceutically acceptable carrier or excipient.

49. A method for forming an injectable, biodegradable neuroscaffold in situ via Michael-type addition comprising:
combining a first suspension of microparticles, nanoparticles, linker moieties, spacer moieties, or any combination thereof, comprising at least two terminal functional alkene group moieties with a second suspension of microparticles, nanoparticles, linker moieties, spacer moieties, or any combination thereof, comprising at least two terminal functional thiol group moieties within a subject, thereby permitting the terminal functional groups of the first suspension to form covalent bonds with the terminal functional groups of the second suspension via a Michael-type addition mechanism in order to yield a self-assembled, covalently cross-linked neuroscaffold with controllable mechanical properties,
provided that at least one of the first suspension or the second suspension comprises microparticles, nanoparticles, or a combination thereof,
wherein the resulting neuroscaffold comprises at least one reducible, hydrolytically cleavable, or enzymatically cleavable bond under physiologically relevant conditions.

50. The method of claim 49, wherein the biodegradable, polymeric microparticles, nanoparticles, or a combination thereof, comprise poly(lactide-co-glycolides) (PLGA), poly(lactides) (PLA), poly(glycolides) (PGA), copolymers of PLGA, PLA, or PGA and a poly(ethylene glycol) (PEG) having a molecular weight of up to 10,000 g/mol, or any combination thereof.

51. The method of claim 50, wherein the PEG copolymer comprises either a terminal functional group moiety that is capable of undergoing a covalent cross-linking reaction via Michael-type addition or a capping group.

52. The method of claim 51, wherein the capping group comprises a primary amine, a carboxyl, a hydroxyl, or a methoxy.

53. The method of claim 49, wherein the terminal functional group moiety capable of undergoing covalent cross-linking via Michael-type addition comprises an alkene, an enone, an acrylate, a vinyl sulfone, a maleimide, or a thiol.

54. The method of claim 49, wherein the covalent cross-linking reaction via Michael-type addition is performed in the presence of a physiologically relevant reducing agent.

55. The method of claim 54, wherein the physiologically relevant reducing agent is glutathione.

56. The method of claim 49, wherein the functionalized linker or spacer moiety is a linear PEG, a multi-arm PEG, a branched PEG, a copolymer of PLGA and PEG, a copolymer of PLA and PEG, a copolymer of PGA and PEG, or any combination thereof, comprising at least two terminal functional group moieties capable of undergoing covalent cross-linking reaction via Michael-type addition.

57. The method of claim 56, wherein the PEG has a molecular weight of up to 10,000 g/mol.

58. The method of claim 56, wherein the terminal functional group moieties capable of undergoing a covalent cross-linking reaction via Michael-type addition are alkenes, enones, vinyl sulfones, maleimides, thiols, or any combination thereof.

59. The method of claim 49, wherein the microparticles, nanoparticles, or a combination thereof, are fabricated by emulsification, precipitation, nanoprecipitation, spray drying, or any combination thereof.

60. The method of claim 49, wherein the microparticles, nanoparticles, or a combination thereof, further comprise one or more agents.

61. The method of claim 60, wherein the one or more agents is a small-molecule, an inhibitor, a peptide, a protein, an antibody, a growth factor, a cytokine, a chemokine, a neurotrophic factor, an oligonucleotide, or any combination thereof.

62. The method of claim 60, wherein the one or more agents is incorporated within the microparticles or nanoparticles, exposed on the surface of the microparticles or nanoparticles, or any combination thereof.

63. The method of claim 60, wherein the one or more agents is incorporated within the neuroscaffold formed in situ, exposed on the surface of the neuroscaffold formed in situ, or any combination thereof.

64. The method of claim 49, wherein the in situ self-assembly is performed in the presence of one or more agents and/or transplantable cells.

65. The method of claim 49, wherein the porosity ranges from nanoporous, having pore sizes of at least 1 nanometer and up to 1000 nanometers, to microporous, having pore sizes of up to 500 microns.

66. The method of claim 49, wherein the mechanical properties are selected and controlled according to the neuroanatomical tissue of interest.

67. The method of claim 49, wherein biodegradation of 50% of the in situ formed neuroscaffold occurs between the time of formation and about 1 hour, 2 hours, 3 hours, 4 hours, 6 hours, 8 hours, 10 hours, 12 hours, 16 hours, 20 hours, 1 day, 2 days, 3 days, 4 days, 5 days, 6 days, 7 days, 8 days, 9 days, 10 days, 12 days, 14 days, 16 days, 18 days, 21 days, 24 days, 28 days, 35 days, 42 days, 49 days, 2 months, 3 months, 4 months, 5 months, 6 months, 7 months, 8 months, 9 months, 10 months, 11 months, 12 months, 14 months, 16 months, 18 months, 21 months, or 24 months, inclusive, post-formation.

68. The method of claim 49, wherein the in situ formed neuroscaffold releases less than 60% of the one or more agents between the time of injection and about 1 hour, 2 hours, 3 hours, 4 hours, 6 hours, 8 hours, 10 hours, 12 hours, 16 hours, 20 hours, 1 day, 2 days, 3 days, 4 days, 5 days, 6 days, 7 days, 8 days, 9 days, 10 days, 12 days, 14 days, 16 days, 18 days, 21 days, 24 days, 28 days, 35 days, 42 days, 49 days, 2 months, 3 months, 4 months, 5 months, 6 months, 7 months, 8 months, 9 months, 10 months, 11 months, 12 months, 14 months, 16 months, 18 months, 21 months, or 24 months, inclusive, post-injection.

69. The method of claim 49, wherein the in situ formed neuroscaffold provides a therapeutically efficacious dose of one or more agents from the time of injection to about 1 hour, 2 hours, 3 hours, 4 hours, 6 hours, 8 hours, 10 hours, 12 hours, 16 hours, 20 hours, 1 day, 2 days, 3 days, 4 days, 5 days, 6 days, 7 days, 8 days, 9 days, 10 days, 12 days, 14 days, 16 days, 18 days, 21 days, 24 days, 28 days, 35 days, 42 days, 49 days, 2 months, 3 months, 4 months, 5 months, 6 months, 7 months, 8 months, 9 months, 10 months, 11 months, 12 months, 14 months, 16 months, 18 months, 21 months, or 24 months, inclusive, post-injection.

70. The method of claim 49, further comprising a pharmaceutically acceptable carrier or excipient.

71. A method of treating a subject having a spinal cord injury or a focal neurological disorder comprising administering to said subject the injectable, biodegradable neuroscaffold of claim 1.

72. A method of treating a subject having a spinal cord injury or a focal neurological disorder comprising administering to said subject an injectable, biodegradable neuroscaffold that is formed in situ by the method of claim 27.

73. A method of treating a subject having a spinal cord injury or a focal neurological disorder comprising administering to said subject an injectable, biodegradable neuroscaffold that is formed in situ by the method of claim 49.

74. The method of claim 71, wherein the focal neurological disorder is caused by nociceptive pain, neuropathic pain, neurotrauma, neuro-inflammation, neurodegenerative diseases, seizure disorders, neurological autoimmune disorders, neuro-oncological diseases, or any combination thereof

75. The method of claim 71, wherein the injectable, biodegradable neuroscaffold is administered to the spinal cord of the subject.

76. The method of claim 75, wherein the injectable, biodegradable neuroscaffold is administered by direct injection into the spinal cord.

77. The method of claim 75, wherein the injectable, biodegradable neuroscaffold is administered by direct injection within close proximity of the spinal cord.

78. The method of claim 71, wherein the injectable, biodegradable neuroscaffold is administered to the identified neuroanatomical or neurophysiological focal site or focal lesion characteristic of the focal neurological disorder.

79. A kit comprising:
a first suspension of microparticles, nanoparticles, linker moieties, spacer moieties, or any combination thereof, comprising at least two terminal functional alkyne group moieties;
a second suspension of microparticles, nanoparticles, linker moieties, spacer moieties, or any combination thereof, comprising at least two terminal functional azide group moieties; and,
instructions for introducing the first and second suspensions into a common location within a subject;
wherein the terminal functional groups of the first suspension and the terminal functional groups of the second suspension covalently bond via a copper-free azide-alkyne cyclo-addition mechanism in order to yield a self-assembled, covalently cross-linked neuroscaffold,
wherein the resulting neuroscaffold comprises at least one reducible, hydrolytically cleavable, or enzymatically cleavable bond under physiologically relevant conditions; and,
wherein at least one of the first suspension or the second suspension comprises microparticles, nanoparticles, or a combination thereof

80. A kit comprising:
a first suspension of microparticles, nanoparticles, linker moieties, spacer moieties, or any combination thereof, comprising at least two terminal functional alkene or trans-cyclooctene group moieties;
a second suspension of microparticles, nanoparticles, linker moieties, spacer moieties, or any combination thereof, comprising at least two terminal functional tetrazine group moieties; and,
instructions for introducing the first and second suspensions into a common location within a subject;
wherein the terminal functional groups of the first suspension and the terminal functional groups of the second suspension covalently bond via a copper-free tetrazine-alkene ligation in order to yield a self-assembled, covalently cross-linked neuroscaffold,
wherein the resulting neuroscaffold comprises at least one reducible, hydrolytically cleavable, or enzymatically cleavable bond under physiologically relevant conditions; and,
wherein at least one of the first suspension or the second suspension comprises microparticles, nanoparticles, or a combination thereof

81. A kit comprising:
a first suspension of microparticles, nanoparticles, linker moieties, spacer moieties, or any combination thereof, comprising at least two terminal functional alkene group moieties;
a second suspension of microparticles, nanoparticles, linker moieties, spacer moieties, or any combination thereof, comprising at least two terminal functional thiol group moieties in the presence of a physiological relevant reducing agent; and,
instructions for introducing the first and second suspensions into a common location within a subject;
wherein the terminal functional groups of the first suspension and the terminal functional groups of the second suspension covalently bond via a Michael-type addition mechanism in order to yield a self-assembled, covalently cross-linked neuroscaffold,
wherein the resulting neuroscaffold comprises at least one reducible, hydrolytically cleavable, or enzymatically cleavable bond under physiologically relevant conditions; and,
wherein at least one of the first suspension or the second suspension comprises microparticles, nanoparticles, or a combination thereof.

82. An injectable, biodegradable neuroscaffold that is formed by the method according to claim 27.

83. An injectable, biodegradable neuroscaffold that is formed by the method according to claim 49.
